(19) United States
(12) Patent Application Publication
(10) Pub. No.: US 2025/0314563 A1
(43) Pub. Date: Oct. 9, 2025

(54) CHAMBER FOR INSPECTING LIQUID SUBSTANCE

(71) Applicant: INTIN INC., Daegu-si (KR)

(72) Inventor: Ji Hoon KIM, Seoul (KR)

(73) Assignee: INTIN INC., Daegu-si (KR)

(21) Appl. No.: 18/962,071

(22) Filed: Nov. 27, 2024

(30) Foreign Application Priority Data
Apr. 5, 2024 (KR) 10-2024-0046801

(51) Int. Cl. G01N 1/14; G01N 1/00

(52) U.S. Cl. CPC G01N 1/14; G01N 2001/002; G01N 2001/1463

(57) ABSTRACT

Disclosed is a chamber for inspecting a liquid substance, the chamber being inserted into a tester for use, and the chamber includes: a chamber sheet comprising a sheet body, an injection groove, and an air outlet formed in a longitudinal direction in the sheet body; and a chamber plate comprising a grip portion and an inserting portion extending in the longitudinal direction from the grip portion. A fastening space to receive and fasten the chamber sheet is formed as a recess in the insertion portion, and wherein where a sample section in which a liquid substance sample is injected and retained is formed in the fastening space.

### CROSS-REFERENCE TO RELATED APPLICATIONS

This application claims priority to and the benefit of Korean Patent Application No. 10-2024-0046801, filed on Apr. 5, 2024, the disclosure of which is incorporated herein by reference in its entirety.

### FIELD

The present disclosure relates to a chamber for inspecting a liquid substance, and more specifically, to a chamber that enables the optimal collection of liquid substance sample to improve the accuracy and ease of liquid substance inspection.

### BACKGROUND

First, the term “liquid substance” hereinafter encompasses all types of bodily fluids found in the human body, such as saliva, blood, semen, sweat, urine, and tears. More broadly, the term “liquid substance” is defined to include various similar liquid substances, including organic and inorganic compounds, used in scientific research fields such as medicine, biotechnology, water quality, and the environment.

Currently, individuals check their health or physical condition by using professional medical institutions or self-testing devices or testers that utilize liquid substances. The use of simple testing devices, which have been in use since the past, remains consistent, and their usage among the younger generation is steadily increasing due to convenience, time and cost efficiency, and quick result confirmation.

Testing devices for assessing an individual's health or physical condition typically use bodily fluids such as saliva, blood, semen, urine, tears, and sweat. Specifically, such testing devices are used to detect hormones contained in the fluid or monitor changes in the condition of the fluid. Representative examples of testing devices include those commonly used for pregnancy tests, diabetes diagnosis, and blood sugar measurements. Additionally, testing devices for liquid substances containing organic and inorganic compounds are being developed and utilized in fields such as medicine, biotechnology, water quality analysis, and environmental studies.

However, with conventional testing devices, it is challenging to accurately control the amount of liquid substance to be measured and efficiently maintain the sample in an optimal state. Consequently, there is a drawback of reduced accuracy when performing liquid substance tests using these devices.

Recently, testing devices have become electronic, enabling a broader range of test items compared to conventional methods. However, a wide variety of electronic testing devices makes it challenging to select a sample chamber designed to fit each device, and operating the testing device is often cumbersome, highlighting the need for a solution to address these issues.

### RELATED LITERATURE

### Patent Document

Korean Patent No. 10-2288223 (Registration Date: Aug. 4, 2021)

### SUMMARY

In view of the above, the present disclosure provides a chamber for inspecting a liquid substance, which collects a liquid substance sample to be inspected in an optimal condition and facilitates easy insertion and removal from a tester.

In one aspect, there is provided a chamber for inspecting a liquid substance, the chamber being inserted into a tester for use, and the chamber includes: a chamber sheet comprising a sheet body, an injection groove, and an air outlet formed in a longitudinal direction in the sheet body; and a chamber plate comprising a grip portion and an inserting portion extending in the longitudinal direction from the grip portion. A fastening space to receive and fasten the chamber sheet is formed as a recess in the insertion portion, and wherein where a sample section in which a liquid substance sample is injected and retained is formed in the fastening space. The sample section includes: an injection slope formed such that one end closer to the grip portion has a predetermined height and creates an inclined surface whose height decreases along the longitudinal direction, with at least a portion inserted into the injection groove; a retention area formed extending in the longitudinal direction on the injection slope and configured to retain the liquid sample; and an observation area formed within the retention area, wherein the liquid substance sample is observed through a lens of the tester.

The chamber for inspecting a liquid substance may further include a double-sided tape adhered to a rear surface of the chamber sheet, wherein, when the chamber sheet is inserted and fastened into the fastening space, the double-sided tape is adhered to a bottom surface of the fastening space but not to the sample section, thereby forming a predetermined space between the retention area and the chamber sheet.

The above double-sided tape may surround outer edges on three sides of the retention area, except for an outer edge connected to the injection slope.

The chamber sheet may have at least two fastening holes formed therein, at least two fastening rods corresponding to the fastening holes may be formed in the fastening space of the chamber plate, and the fastening rods may be inserted through the fastening holes such that portions of ends of the fastening rods are exposed outside the fastening holes.

The chamber sheet may be fastened into the fastening space, each of the exposed portions may be pressed externally to spread wider than a diameter of each of the fastening holes, thereby securing the chamber sheet to the chamber plate.

The above sample section may further include a retention confirmation area which is connected to the retention area through a microchannel, and which has a color-changing material that changes color upon contact with the liquid substance sample.

The grip portion may be exposed to the outside of the tester while the liquid substance testing chamber is inserted into the tester and secured.

The chamber plate may include: a guide protrusion protruding by a predetermined length in the longitudinal direction without encroaching on the fastening space; and a guide groove recessed by a predetermined length in the longitudinal direction, wherein both the guide protrusion and the guide groove are formed in an upper surface of the insertion portion.

A fastening confirmation part may be formed to protrude in a predetermined shape on the rear surface of the chamber plate. When the chamber for inspecting a liquid substance is fully inserted into the tester, the fastening confirmation part may engage with a groove formed at an entrance of the tester, allowing a user to visually confirm completion of the insertion.

The chamber plate may have cutting surfaces formed by cutting side and rear surface thereof from a first end of the insertion portion by a predetermined length. The cut surfaces may be identically formed on both side surfaces of the insertion portion, starting with a widest width near the first end and gradually narrowing in the longitudinal direction.

The insertion portion may be formed at the first end of the insertion portion to prevent the chamber from being inserted any further when the chamber is fully inserted into the tester.

The stopper may be in a shape of a groove formed from the first end toward the fastening space, and the groove may be formed by a predetermined length in the X-axis direction with an inner width that is narrowest, then the width of the groove may gradually increase and narrow again near the first end, and a sound may be generated when the chamber is fully inserted into the tester.

The double-sided tape may have at least one color other than white.

### BRIEF DESCRIPTION OF THE DRAWINGS

FIG. 1 is a side perspective view of a chamber for inspecting a liquid substance according to an embodiment of the present disclosure.

FIG. 2 is an exploded perspective view of a chamber for inspecting a liquid substance according to an embodiment of the present disclosure.

FIG. 3 is a drawing for explaining a sample section according to an embodiment of the present disclosure.

FIG. 4 is a perspective view of a chamber sheet according to an embodiment of the present disclosure.

FIG. 5 is a longitudinal cross-sectional view of a chamber according to an embodiment of the present disclosure.

FIG. 6 is a top perspective view of a chamber plate according to an embodiment of the present disclosure.

FIG. 7 is a rear perspective view of a chamber plate according to an embodiment of the present disclosure.

FIG. 8 is a front view showing a state in which a chamber for inspecting a liquid substance according to an embodiment of the present disclosure is connected to a tester.

FIG. 9 is a rear view showing a state in which a chamber for inspecting a liquid substance according to an embodiment of the present disclosure is connected to a tester.

### DETAILED DESCRIPTION

Description will now be given in detail according to exemplary embodiments disclosed herein, with reference to the accompanying drawings. For the sake of brief description with reference to the drawings, the same or equivalent components may be provided with the same or similar reference numbers, and description thereof will not be repeated. In addition, in the following description of the embodiments, a detailed description of known functions and configurations incorporated herein will be omitted when it may impede the understanding of the embodiments.

While terms including ordinal numbers, such as “first” and “second,” etc., may be used to describe various components, such components are not limited by the above terms. The above terms are used only to distinguish one component from another.

The singular forms are intended to include the plural forms, unless the context clearly indicates a different meaning.

In this application, the described steps may be carried out in any sequence, except in cases where a clearly defined cause-and-effect relationship necessitates a specific order.

It will be further understood that the terms “comprise”, “include”, “have”, etc. when used in this specification, specify the presence of stated features, integers, steps, operations, elements, components, and/or combinations of them but do not preclude the presence or addition of one or more other features, integers, steps, operations, elements, components, and/or combinations thereof.

Hereinafter, the present disclosure will be described with reference to the attached drawings.

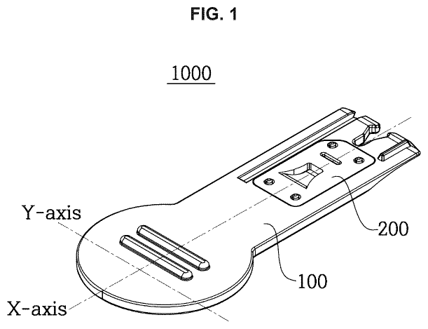

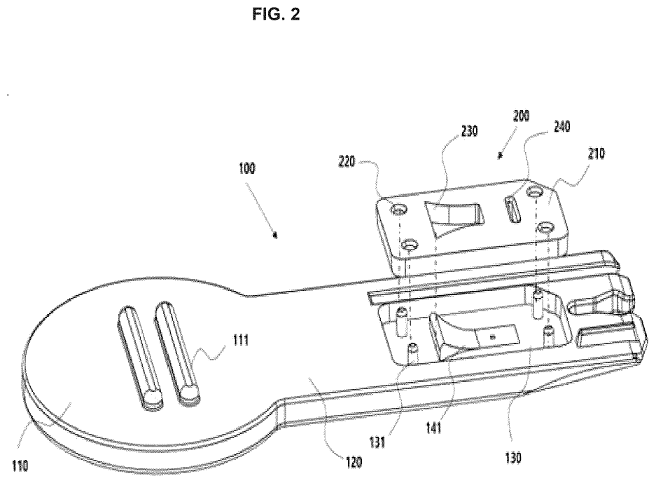

FIG. 1 is a side perspective view of a chamber for inspecting a liquid substance according to an embodiment of the present disclosure, and FIG. 2 is an exploded perspective view of a chamber for inspecting a liquid substance according to an embodiment of the present disclosure.

Referring to FIGS. 1 and 2, a chamber 1000 for inspecting a liquid substance according to an embodiment of the present disclosure may include a chamber plate 100 and a chamber sheet 200.

The chamber plate 100 may include a grip portion 110 and an inserting portion 120 extending in the x-axis direction, that is, in a longitudinal direction, from the grip portion 110.

In the insertion portion 120, a fastening space 130 into which a chamber sheet 200 is inserted and fastened is formed in the longitudinal direction, that is, the X-axis direction, and the fastening space 130 is shaped as a groove with a predetermined depth. It is preferable that the predetermined depth of the fastening space 130 be such that an upper surface of the chamber plate 100 and an upper surface of the chamber sheet 200 become flush with each other when the chamber sheet 200 is inserted into the fastening space 130. The overall shape of the insert 120 has the same shape as the chamber sheet 200.

In the bonding space 130, a retention area 140 in which a liquid substance sample is introduced and retained is formed in the longitudinal direction. That is, an injection slope 141 of the retention area 140 is formed in the longitudinal direction, and a retention area 142 (see FIG. 5) is formed extending in the longitudinal direction of the injection slope 141. Here, the formation of the retention area 140 in the longitudinal direction means that a liquid substance sample is supplied to the injection slope 141 in the longitudinal direction.

The grip portion 110 has a width greater than the width (i.e., a length in the Y-axis direction) of the insertion portion 120, and allows the user to push the grip portion 110 for insertion into a tester 2000 and pull the grip portion 110 for removal from the tester 2000. In addition, at least one anti-slip protrusion 111 may be formed in the grip portion 110 to prevent a user's hand from slipping. At least one anti-slip protrusion 111 may be formed in the Y-axis direction, which is perpendicular to the longitudinal direction.

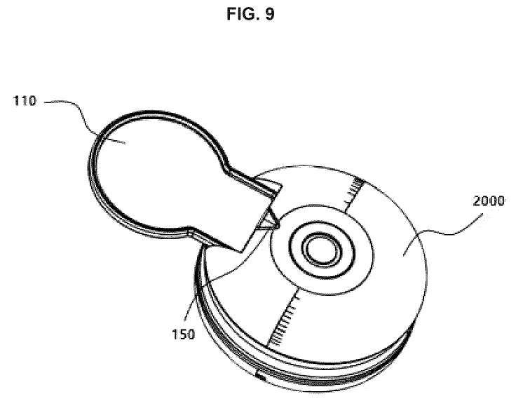

In FIGS. 1 and 2, the grip portion 110 is illustrated as circular, but it may be manufactured in various shapes. Referring to FIG. 8, when the chamber plate 100 is inserted into the tester 2000, the grip portion 110 is not inserted into the tester 2000 but remains outside the tester 2000, allowing the user to easily hold the tester 2000 by hand.

While containing a liquid substance sample, the chamber plate 100 may be partially inserted into a liquid substance testing device 2000 (see FIG. 8), thereby providing the liquid substance sample to the tester 2000.

The chamber plate 100 may be formed of a wide range of materials that may be commonly used in testers in the relevant technical field to form a chamber for inspecting a liquid substance. However, the chamber plate 100 may be preferably formed of a light-transmitting material that allows light to pass through.

The light-transmitting material may include materials commonly used in the relevant technical field to form a base substrate. For the light-transmitting material, transparent materials may be used to enhance light transmittance and achieve a clear observation screen. For example, the light-transmitting material may be formed of one of transparent synthetic resins (for example, polycarbonate, polyethylene, and acrylic), natural rubber, and glass, but is not limited thereto.

The chamber sheet 200 may include a sheet body 210, a fastening hole 220, an injection groove 230, and an air outlet 240.

The sheet body 210 has the same shape as that of the fastening space 130. If the sheet body 210 has at least one vertex, each vertex may be chamfered and shaped into a curve to allow easy insertion into the fastening space 130.

The fastening space 130 and the sheet body 210 may be configured such that at least one of the edges or vertices is modified in shape. In this case, if an upper surface of the sheet body 210 is exposed and each configuration 220 and 230 are properly aligned and fastened to the fastening space 130, the fastening is completed. Conversely, if a rear surface of the sheet body 210 is exposed and fastened to the fastening space 130, fastening does not occur. Referring to FIGS. 1 and 2, the fastening space 130 and the sheet body 210 are manufactured in a square shape, with one of the corners of the square diagonally cut. This ensures that fastening is only possible when the upper surface of the sheet body 210 is exposed and inserted in a direction that aligns with the arrangement of Each configuration of the sample section 140.

The fastening hole 220 is provided as at least two holes. The fastening hole 220 serve as a hole into which a fastening rod 131 formed in the fastening space 130 is inserted. The fastening hole 220 and the fastening rod 131 may be omitted when the chamber sheet 200 is secured to the fastening space 130 by press fitting, screwing, laser welding, welding, or taping. Each fastening rod 131 is inserted into one of a plurality of fastening holes 220, with a portion of the end of the corresponding fastening rod 131 exposed outside the fastening hole 220. The exposed portion of the corresponding fastening rod 131 is pressed by equipment, causing the fastening rod 131 to spread wider than a diameter of the fastening hole 220. This prevents the chamber sheet 200 from detaching from the chamber plate 100, even under external forces.

The injection groove 230 is a hole that allows a liquid substance sample to be delivered to the injection slope 141 of the sample section 140 while the chamber sheet 200 is inserted and fastened into the fastening space 130. The injection groove 230 is shaped to conform to the injection slope 141. At least a portion of the injection slope 141 may be inserted into the injection groove 230 when the chamber sheet 200 is inserted into the fastening space 130. Preferably, a protruding portion of the injection slope 141 may fit snugly into the injection groove 230.

The air outlet 240 is a hole for exhausting air from the retention area 142. Due to the air outlet 240, external air that may unintentionally enter the retention area 142 and interfere with the observation and analysis of a liquid substance sample may be expelled, thereby enhancing the accuracy of a liquid substance test.

Meanwhile, the chamber sheet 200 may be fastened to the fastening space 130 using various conventional physical fastening methods, in addition to the above-described fastening technique.

The chamber sheet 200 may be formed of a wide range of materials commonly used in testers in the relevant technical field to form a chamber for inspecting a liquid substance. Preferably, the chamber sheet 200 may be formed of a light-transmitting material that allows the same light to pass through as the chamber plate 100.

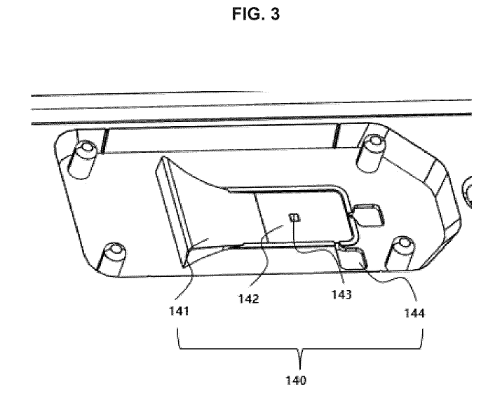

FIG. 3 is a drawing for explaining a sample section according to an embodiment of the present disclosure. Referring to FIG. 3, the sample section 140 is a space where a liquid substance to be inspected is applied and serves to hold a liquid substance sample so that a camera lens of the tester 2000 can capture the liquid substance.

The sample section 140 is formed within the fastening space 130 of the insertion portion 120 and may easily hold a liquid substance sample. The sample section 140 may include an injection slope 141, a retention area 142, and an observation area 143. In some cases, the sample section 140 may further include a retention confirmation area 144.

The injection slope 141 is formed in the longitudinal direction of the chamber plate 100 to facilitate the easy injection of a liquid substance sample. One end of the injection slope 141, located near the grip portion 110, may have a predetermined height that progressively decrease along the longitudinal direction, forming a sloped surface. Therefore, the liquid substance sample is first injected into the highest end of the injection slope 141 and then flows along the sloped surface in the longitudinal direction.

The retention area 142 extends along the injection slope 141 in the longitudinal direction and holds a liquid substance sample supplied along the inclined surface of the injection slope 141. The retention area 142 is formed flat to ensure the liquid substance sample is evenly distributed. In some cases, the retention area 142 may be recessed to a predetermined depth or formed with a protective wall of a predetermined height along an outer boundary of the retention area 142.

The observation area 143 is an area where the liquid substance sample supplied to the retention area 142 is collected and observed through the lens of the tester 2000. It is preferable for the observation area 143 to be square-shaped, but the observation area 143 may be formed in various other shapes. The observation area 143 may be formed as a recessed shape on a bottom surface of the sample section 140. It is preferable for the observation area 143 to be formed near the center of the retention area 142, but the position of the observation area 143 may vary depending on the case.

As the injection slope 141 and the retention area 142 are formed in the same direction as the longitudinal direction of the chamber plate 100, the liquid substance sample may be more easily injected into the sample section 140 and provided in an appropriate amount and optimal condition, thereby improving the accuracy and convenience of liquid substance inspection.

Each configuration 141 to 143 of the sample section 140 may be formed of a wide range of materials commonly used in testers in the relevant technical field to facilitate the retention and observation of a liquid substance sample. Preferably, each configuration 141 to 143 of the sample section 140 may be formed of a light-transmitting material that allows light to pass through.

Additionally, each configuration 141 to 143 of the sample section 140 may be formed of a material which is the same as or different from that of the chamber plate 100.

The retention confirmation area 144 may be formed within or outside the retention area 142. When formed outside the retention area 142, the retention confirmation area 144 may be connected to the retention area 142 through a microchannel. The retention confirmation area 144 includes a color-changing material that reacts to a liquid substance sample by changing color. This color-changing material may include litmus paper or the like.

After a liquid substance sample is injected into the sample section 140, the retention confirmation area 144 allows the user to visually confirm, using the color-changing material, whether the liquid substance sample has been properly supplied. Accordingly, if the retention confirmation area 144 is formed, the retention confirmation area 144 should remain open and not sealed.

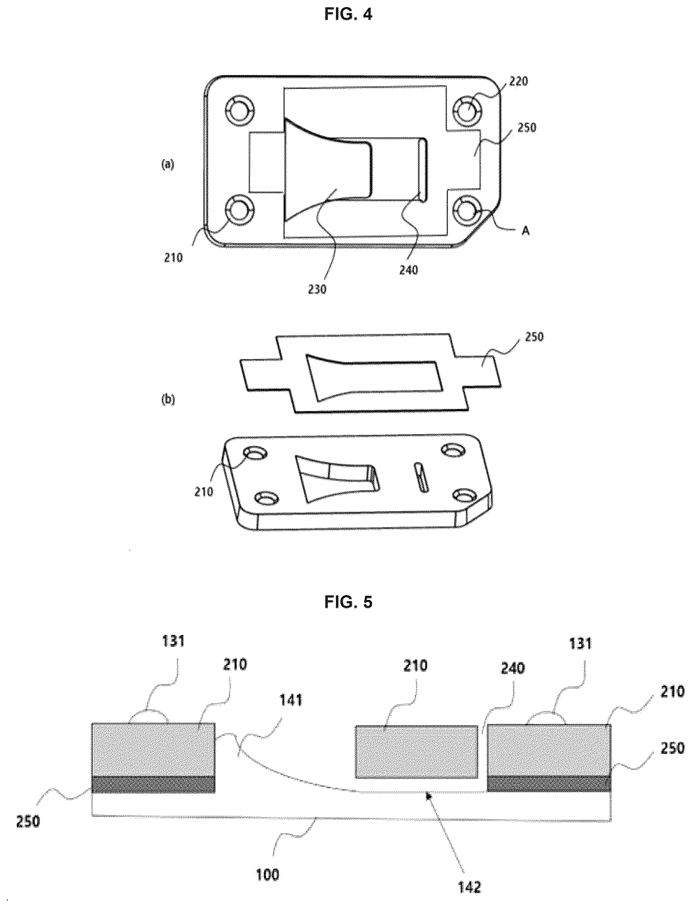

FIG. 4 is a perspective view of a chamber sheet according to an embodiment of the present disclosure, wherein (a) is a perspective view showing a rear surface of the chamber sheet 200, and (b) is an exploded perspective view of the rear surface of the chamber sheet 200.

Referring to FIG. 4, the chamber sheet 200 may have a double-sided tape 250 attached to the rear surface thereof. The double-sided tape 250 is adhered to the rear surface of the chamber sheet 200 in a shape that exposes each fastening hole 220, the injection groove 230, the air outlet 240, and a space between the air outlet 240 and the injection groove 230. In other words, the double-sided tape 250 is adhered to the remaining area of the rear surface of the chamber sheet 200, except for areas where each fastening hole 220, the injection groove 230, the air outlet 240, and the space between the air outlet 240 and the injection groove 230 are formed. If the retention confirmation area 144 is configured, the double-sided tape 250 may be shaped to expose an area of the chamber sheet 200 corresponding to the retention confirmation area 144.

The double-sided tape 250 may be adhered to a bottom surface of the fastening space 130 when the chamber sheet 200 is inserted and fastened into the fastening space 130. For example, the double-sided tape 250 may be adhered to the remaining areas of the bottom surface of the fastening space 130, except for the areas where the fastening hole 220, the retention area 142, and the injection slope 141 are formed.

The double-sided tape 250 is manufactured in a color to make the injection groove 230 stand out to a user, and this color may be any color other than white. The color of the double-sided tape 250 is preferably black, but may also be red, yellow, blue, or other colors.

Meanwhile, a gap between the retention area 142 and the chamber sheet 200 may be formed thin enough to allow a liquid substance sample to be evenly distributed for easy observation, and the gap may vary depending on a liquid substance to be inspected. Specifically, the gap between the retention area 142 and the chamber sheet 200 may be determined based on an average size (average diameter) of a measurement target contained in the liquid substance to be inspected.

For example, when measuring sperm cells contained in semen, the average diameter of a sperm cell head is about 5 to 8 μm. Therefore, maintaining the gap between the retention area 142 and the chamber sheet 200 at 10 μm or less has the advantage of preventing multiple sperm cells from stacking in multiple layers. In this case, the sperm cells contained in the semen may be evenly distributed in a single layer, facilitating easy observation and analysis of the sperm cells within the semen sample. As another example, when measuring white blood cells with an average diameter of 16 to 20 μm, it may be advantageous for the gap between the retention area 142 and the chamber sheet 200 to be 30 μm or less.

Accordingly, the gap between the retention area 142 and the chamber sheet 200 may be determined based on the size of the measurement target but preferably should be less than 50 μm. More preferably, the gap between the retention area 142 and the chamber sheet 200 may be less than 30 μm; even more preferably, less than 20 μm; and most preferably, less than 10 μm.

Meanwhile, the gap between the retention area 142 and the chamber sheet 200 is determined by a thickness of the double-sided tape 250. The gap between the retention area 142 and the chamber sheet 200 will be described with reference to FIG. 5. FIG. 5 is a longitudinal cross-sectional view of a chamber according to an embodiment of the present disclosure.

As described above, the chamber sheet 200 is inserted and fastened such that the double-sided tape 250 adhered to the rear surface thereof is adhered to the bottom surface of the fastening space 130. The areas of the chamber sheet 200 where the double-sided tape 250 is not adhered are positioned over the injection slope 141, the retention area 142, and the retention confirmation area 144. That is, the double-sided tape 250 is not adhered over the injection slope 141, the retention area 142, and the retention confirmation area 144.

Accordingly, because the double-sided tape 250 is not adhered over the retention area 142, a space equal to the thickness of the double-sided tape 250 is formed. In other words, a space equal to the thickness of the double-sided tape 250 is formed between the retention area 142 and the sheet body 210 of the chamber sheet 200.

In addition, since the double-sided tape 250 is adhered to the bottom surface of the fastening space 130, except for the retention area 142, the double-sided tape 250 surrounds the outer edges of the retention area 142. Specifically, the double-sided tape 250 surrounds the outer edges on three sides of the retention area 142, except for an outer edge connected to the injection slope 141. As a result, the liquid substance sample held in the retention area 142 is blocked by the double-sided tape 250 and prevented from escaping to the outside.

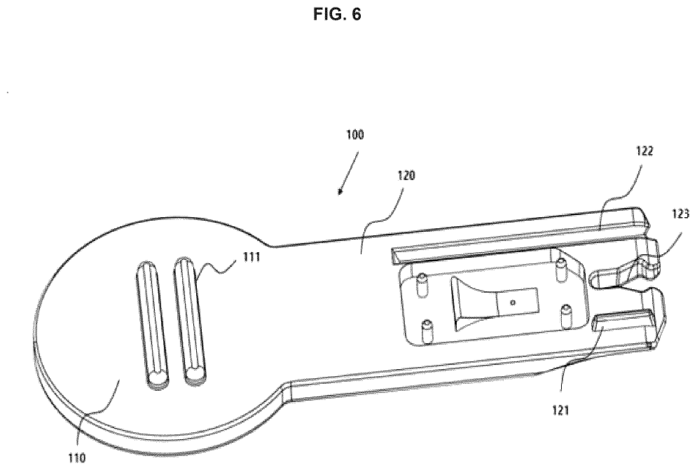

FIG. 6 is a top perspective view of a chamber plate according to an embodiment of the present disclosure. Referring to FIG. 6, the upper surface of the insertion portion 120 of the chamber plate 100 may have a guide protrusion 121 protruding in the longitudinal direction by a predetermined length without encroaching on the fastening space 130, and a guide groove 122 recessed in the longitudinal direction by a predetermined length.

The guide protrusion 121 is seated in a fastening groove (not shown) formed inside the tester 2000, and a projection formed inside the tester 2000 is inserted into the guide groove 122. Accordingly, the guide groove 122 guides an insertion path so that the plate 100 is inserted smoothly, without misalignment, when inserted into the tester 2000.

The guide protrusion 121 and the guide groove 122 are formed spaced apart from each other and designed not to encroach on the fastening space 130. For example, the guide protrusion 121 extends from a first end of the insertion portion 120, which is the end first inserted into the tester 2000, toward the grip portion 110 by a predetermined length in the longitudinal direction. Similarly, the guide groove 122 is formed from the first end toward the grip portion 110 by a predetermined length in the longitudinal direction.

The predetermined lengths of the guide protrusion 121 and the guide groove 122 may be the same or different. In cases where the predetermined lengths differ, as shown in FIG. 6, the predetermined length of the guide groove 122 may be longer than that of the guide protrusion 121. In some cases, the predetermined length of the guide protrusion 121 may be longer than that of the guide groove 122.

While allowing the user to easily insert the chamber 1000 into the tester 2000, these guide protrusion 121 and guide grooves 122 also prevent reverse insertion by blocking the chamber 1000 when the user tries to insert the chamber 1000 with the rear surface facing upward.

Meanwhile, a stopper 123 may be formed at the first end of the insertion portion 120 of the chamber plate 100 to prevent the chamber 1000 from being inserted any further when the chamber 1000 is fully inserted into the tester 2000. The stopper 123 may be formed in the shape of a groove extending from the first end toward the fastening space 130. For example, the stopper 123 may be formed such that a predetermined length is formed in the X-axis direction while a width of an inner groove (which is a length in the Y-axis direction) is narrowest. Then, as the groove progresses, the width of the groove gradually increases and then narrows again near the first end. When the stopper 123 has the above-described shape, a “click” sound occurs when the chamber 1000 is fully inserted into the tester 2000, allowing the user to audibly confirm that the chamber 1000 has been completely inserted.

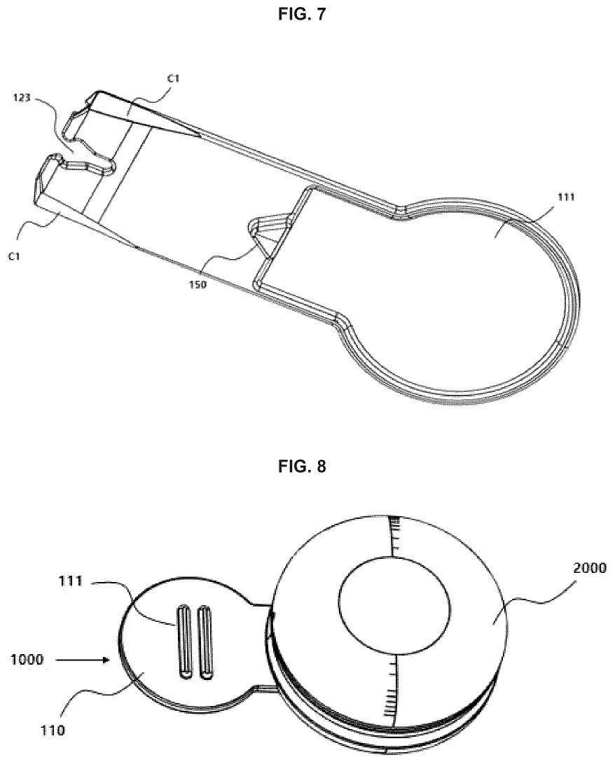

FIG. 7 is a rear perspective view of a chamber plate according to an embodiment of the present disclosure. Referring to FIG. 7, the chamber plate 100 may have cut surfaces C1, which are formed by cutting the side and rear surfaces from the first end of the insertion portion 120 by a predetermined length. The cut surfaces C1 are identically formed on both side surfaces of the insertion portion 120, starting with the widest width near the first end and gradually narrowing.

When the chamber 1000 is inserted, these two cut surfaces C1 prevent a portion of the rear surface with the cut surfaces C1 formed from touching a bottom of an insertion passage in the tester 2000. Instead, the two cut surfaces C1 allows an area of the rear surface without the cut surfaces C1 formed to contact the bottom of the insertion passage. This allows the user to insert the chamber 1000 smoothly without exerting force.

Additionally, a triangular protruding fastening confirmation part 150 may be formed in the rear surface of the chamber plate 100. The fastening confirmation part 150 becomes exposed outside the tester 2000 when the chamber 1000 is fully inserted through the stopper 123. Thus, the user is able to visually confirm a coupling status between the fastening confirmation part 150 and the tester 2000 to verify that insertion is complete. Moreover, the fastening confirmation part 150 may have a reverse insertion prevention function, preventing the chamber 1000 from being fully inserted into the tester 2000 if the user attempts to insert the chamber 1000 upside down. Additionally, the fastening confirmation part 150 may be formed in shapes other than a triangle.

FIG. 8 is a front view showing a state in which a chamber for inspecting a liquid substance according to an embodiment of the present disclosure is attached to a tester, and FIG. 9 is a rear view showing a state in which a chamber for inspecting a liquid substance according to an embodiment of the present disclosure is attached to a tester.

When the chamber 1000 is fully fastened to the tester 2000, the grip portion 110 is exposed outside the tester 2000, and the fastening confirmation part 150 engages with a groove formed at an entrance of the tester 2000 and thus is exposed outside the tester 2000.

For a chamber for inspecting a liquid substance according to the present disclosure and a tester using the same, it is possible to effectively collect a target liquid substance sample in an appropriate amount and optimal condition, facilitating easy insertion into and removal from the tester.

The technical features disclosed in each embodiment of the present disclosure are not limited to a corresponding embodiment, and unless incompatible with each other, the technical features disclosed in each embodiment may be applied in combination to other embodiments.

Therefore, although each embodiment is described mainly about an individual technical feature, the technical features of the embodiments of the present disclosure may be applied in combination, unless incompatible with each other.

The present disclosure is not limited to the above-described embodiments and the accompanying drawings, and various modifications and changes may be made in view of a person skilled in the art to which the present disclosure pertains. Therefore, the scope of the present disclosure should be determined by the scope of the appended claims, and equivalents thereof.

What is claimed is:

1. A chamber for inspecting a liquid substance, the chamber being inserted into a tester for use, the chamber comprising:
a chamber sheet comprising a sheet body, an injection groove, and an air outlet formed in a longitudinal direction in the sheet body; and
a chamber plate comprising a grip portion and an inserting portion extending in the longitudinal direction from the grip portion, wherein a fastening space to receive and fasten the chamber sheet is formed as a recess in the insertion portion, and wherein where a sample section in which a liquid substance sample is injected and retained is formed in the fastening space,
wherein the sample section comprises: an injection slope formed such that one end closer to the grip portion has a predetermined height and creates an inclined surface whose height decreases along the longitudinal direction, with at least a portion inserted into the injection groove; a retention area formed extending in the longitudinal direction on the injection slope and configured to retain the liquid sample; and an observation area formed within the retention area, wherein the liquid substance sample is observed through a lens of the tester.

2. The chamber of claim 1, further comprising a double-sided tape adhered to a rear surface of the chamber sheet, wherein, when the chamber sheet is inserted and fastened into the fastening space, the double-sided tape is adhered to a bottom surface of the fastening space but not to the sample section, thereby forming a predetermined space between the retention area and the chamber sheet.

3. The chamber of claim 2, wherein the double-sided tape surrounds outer edges on three sides of the retention area, except for an outer edge connected to the injection slope.

4. The chamber of claim 1, wherein:
the chamber sheet has at least two fastening holes formed therein,
at least two fastening rods corresponding to the fastening holes are formed in the fastening space of the chamber plate, and
the fastening rods are inserted through the fastening holes such that portions of ends of the fastening rods are exposed outside the fastening holes.

5. The chamber of claim 4, wherein when the chamber sheet is fastened into the fastening space, each of the exposed portions is pressed externally to spread wider than a diameter of each of the fastening holes, thereby securing the chamber sheet to the chamber plate.

6. The chamber of claim 1, wherein the sample section further comprises a retention confirmation area which is connected to the retention area through a microchannel, and which has a color-changing material that changes color upon contact with the liquid substance sample.

7. The chamber of claim 1, wherein the grip portion is exposed to the outside of the tester while the liquid substance testing chamber is inserted into the tester and secured.

8. The chamber of claim 1, wherein the chamber plate comprises: a guide protrusion protruding by a predetermined length in the longitudinal direction without encroaching on the fastening space; and a guide groove recessed by a predetermined length in the longitudinal direction, wherein both the guide protrusion and the guide groove are formed in an upper surface of the insertion portion.

9. The chamber of claim 1, wherein a fastening confirmation part is formed to protrude in a predetermined shape on the rear surface of the chamber plate, and wherein when the chamber for inspecting a liquid substance is fully inserted into the tester, the fastening confirmation part engages with a groove formed at an entrance of the tester, allowing a user to visually confirm completion of the insertion.

10. The chamber of claim 1, wherein the chamber plate has cutting surfaces formed by cutting side and rear surface thereof from a first end of the insertion portion by a predetermined length, and wherein the cut surfaces are identically formed on both side surfaces of the insertion portion, starting with a widest width near the first end and gradually narrowing in the longitudinal direction.

11. The chamber of claim 1, wherein a stopper is formed at the first end of the insertion portion to prevent the chamber from being inserted any further when the chamber is fully inserted into the tester.

12. The chamber of claim 11, wherein the stopper is in a shape of a groove formed from the first end toward the fastening space, and wherein the groove is formed by a predetermined length in the X-axis direction with an inner width that is narrowest, then the width of the groove gradually increases and narrows again near the first end, and a sound is generated when the chamber is fully inserted into the tester.

13. The chamber of claim 2, wherein the double-sided tape has at least one color other than white.
